(19) United States
(12) Patent Application Publication
(10) Pub. No.: US 2002/0197261 A1
(43) Pub. Date: Dec. 26, 2002

(54) Therapeutic agent/ligand conjugate compositions, their methods of synthesis and use

(76) Inventors: Chun Li, Missouri City, TX (US); Javier O. Vega, Houston, TX (US); Shi Ke, Missouri City, TX (US); Sidney Wallace, Bellaire, TX (US)

Correspondence Address: Lori D. Stiffler, Baker Botts L.L.P., One Shell Plaza, 910 Louisiana Street, Houston, TX, 77002-4995, US

(21) Appl. No.: 10/126,369

(22) Filed: Apr. 19, 2002

Related U.S. Application Data
(60) Provisional application No. 60/286,453, filed on Apr. 26, 2001.
(60) Provisional application No. 60/334,969, filed on Dec. 4, 2001.
(60) Provisional application No. 60/343,147, filed on Dec. 20, 2001.

(51) Int. Cl. A61K 39/395; C07K 16/46

(52) U.S. Cl. 424/178.1; 530/391.1

(57) ABSTRACT

Conjugate molecules comprising a ligand or targeting moiety bonded to a polymer spacer, a polymer carrier bonded to the polymer spacer, and a therapeutic agent bound to the polymer carrier (with or without a linker) are disclosed. The conjugate molecules are useful for the selective delivery of therapeutic agents to tumors or other tissues expressing biological receptors.

### CROSS REFERENCE TO RELATED APPLICATIONS

This application claims the benefit of: U.S. Provisional Patent Application No. 60/286,453, entitled “Methods for Visualizing Tumors Using a Radioisotope Conjugate” filed Apr. 26, 2001; U.S. Provisional Patent Application No. 60/334,969, entitled “Therapeutic Agent/Ligand Conjugate Compositions and Methods of Use” filed Dec. 4, 2001; and U.S. Provisional Patent Application No. 60/343,147, entitled “Diagnostic Imaging Compositions, Their Methods of Synthesis and Use” filed Dec. 20, 2001, all three of which are hereby incorporated herein by reference in their entirety. This application is related to U.S. patent application Ser. No. , entitled “Diagnostic Imaging Compositions, Their Methods of Synthesis and Use,” filed Apr. 19, 2002, inventors Chun Li, et al., which is hereby incorporated herein by reference in its entirety.

### RIGHTS IN THE INVENTION

This invention was made, in part, with United States Government support under grant CA 74819 from the NCI, and the United States Government may therefore have certain rights in the invention.

### BACKGROUND OF THE INVENTION

1. Field of the Invention

This invention relates to compositions useful in the treatment of cancer and other diseases, and, more specifically, to compositions comprising therapeutic agents (e.g., chemotherapeutic drugs) and other compounds conjugated to ligands, useful for the selective delivery of the agent or compound to tumors and other target tissues. The invention also relates to methods for synthesizing and using such compositions.

2. Description of the Background

Cancer chemotherapy is ultimately limited by the toxicity of drugs to normal tissues. Selective delivery of drugs to target cells theoretically allows the use of a reduced dose to achieve the same therapeutic response, with a consequent decrease in systemic toxicity. A number of methods have been used to selectively target tumors with therapeutic agents to treat cancers in humans and other animals. Targeting moieties such as monoclonal antibodies (mAb) or their fragments have been conjugated to linear polymers via their side chain functional groups. However, this approach usually results in reduced receptor binding affinity either due to changes in the chemical properties of the antibodies or due to folded configuration of polymers that imbed the targeting moiety in the random coiled structure. Moreover, crosslinks and aggregates of polymers may form as a result of side-chain coupling procedures. Immunoconjugates have been synthesized by employing intermediate carriers such as dextran, serum albumin, and synthetic polymers to increase the amount of drugs attached to the antibody without significantly impairing its antigen binding activity. However, in these cases, the antibodies were attached to the side chains of the polymer, which is believed to adversely affect the binding affinity of the antibody and the in vivo behavior of the immunoconjugates.

Thus, there exists a need for new and improved compositions and methods for the treatment of tumors and other diseases.

### SUMMARY OF THE INVENTION

The present invention overcomes problems and disadvantages associated with current therapeutic agents, and provides novel compositions for treatment of tumors and other diseases. Preferred embodiments allow for the selective delivery of a therapeutic agent (e.g., a chemotherapeutic agent) or another compound or agent to the target tumor or tissue. Compositions according the invention include conjugates of a ligand, a polymer spacer, a polymer carrier, and a therapeutic agent or another compound or agent. A preferred composition of the invention comprises a conjugate of an antibody, a polyethylene glycol (PEG) spacer, a polymer carrier, and a therapeutic agent. In a particularly preferred embodiment, the ligand is a monoclonal antibody, the polymer spacer is a PEG spacer, the polymer carrier is poly(1-glutamic acid) (PG), and the therapeutic agent is a chemotherapeutic agent such as Adriamycin or paclitaxel.

Accordingly, one embodiment of the invention is directed to a conjugate molecule comprising: a ligand; a polymer spacer; a polymer carrier; and a therapeutic agent. The ligand is bonded to the polymer spacer, the polymer spacer is bonded to the polymer carrier, and the polymer carrier is bonded to the therapeutic agent. The polymer carrier may be bonded to the therapeutic agent with or without the assistance of a linker molecule.

Another embodiment of the invention is directed to a composition comprising any of the conjugate molecules described herein and a pharmaceutically acceptable carrier.

Still another embodiment is directed to a method for selectively delivering a therapeutic agent to a target tissue in a patient comprising: administering a conjugate molecule to the patient having said target tissue, wherein the conjugate molecule comprises: a ligand with affinity for the target tissue; a polymer spacer; a polymer carrier; and a therapeutic agent. The ligand is bonded to the polymer spacer, the polymer spacer is bonded to the polymer carrier, and the polymer carrier is bonded to the therapeutic agent.

A further embodiment is directed to a method of treating a patient having a diseased tissue, the method comprising administering a therapeutically effective amount of a conjugate molecule to the patient, wherein the conjugate molecule comprises: a ligand with affinity for the diseased tissue; a polymer spacer; a polymer carrier; and a therapeutic agent. The ligand is bonded to the polymer spacer, the polymer spacer is bonded to the polymer carrier, and the polymer carrier is bonded to the therapeutic agent.

The invention also includes different methods for synthesizing conjugate molecules of the invention. One such method comprises the steps of: providing a polymer spacer-polymer carrier construct having a sulfhydryl-reactive vinyl sulfone group at one end of the polymer spacer; conjugating the therapeutic agent to the polymer carrier to form a vinyl sulfone-polymer spacer-polymer carrier-therapeutic agent construct; pretreating the ligand to introduce a sulfhydryl group on the ligand; and combining the ligand with the vinyl sulfone-polymer spacer-polymer carrier-therapeutic agent construct, wherein the vinyl sulfone group reacts with the sulfhydryl group to form a conjugate molecule comprising the ligand, the polymer spacer, the polymer carrier, and the therapeutic agent, and wherein the ligand is bonded to the polymer spacer, the polymer spacer is bonded to the polymer carrier, and the polymer carrier is bonded to the therapeutic agent.

Other objects and advantages of the invention are set forth in part in the description which follows, and, in part, will be obvious from this description, or may be learned from the practice of the invention.

### DESCRIPTION OF THE FIGURES

The following figures form part of the present specification and are included to further demonstrate certain aspects of the present invention. The invention may be better understood by reference to one or more of these drawings in combination with the detailed description of the specific embodiments presented herein.

FIG. 1. Schematics of conjugate molecules depicting site-specific attachment of homing ligand to one terminus of PEG molecules for targeted delivery of diagnostic and therapeutic agents.

FIG. 2. Synthetic scheme for the synthesis of mAb-PEG-PG-Drug conjugates.

FIG. 3. Comparison of GPC chromatograms of VS-PEG-PG conjugate (B) and VS-PEG (A).

FIG. 4. ¹H-NMR of VS-PEG-PG.

FIG. 5. Structure of Adriamycin.

FIG. 6. GPC elution profile of Herceptin (A), VS-PEG-PG-TXL (B), and purified Herceptin-PEG-PG-TXL conjugate (C) using a Superdex 200 column (1.0×30 cm).

FIG. 7. Purification of C225-PEG-PG-Adr- by FPLC using a Resource Q anion-exchange column. Fractions 3-5 correspond to C225; fractions 14-21 correspond to C225-PEG-PG-Adr conjugate.

FIG. 8. Gel permeation chromatography of C225 (A), PEG-PG-Adr (B), and purified C225-PEG-PG-Adr conjugate (C) using a Superdex 200 column (1.0×30 cm).

FIG. 9. Volume-weighted Gaussian Analysis showing particle size and size distribution of C225-PEG-PG-Adr.

FIG. 10. Hypothetical structure of polymeric nanoparticles (A) and targetable polymeric nanoparticles (B) from amphiphilic block copolymer PEG-PG-Adr.

FIG. 11. Graphs showing cytotoxicity of Herceptin-PEG-PG-TXL in MDA-MB-468 (Her 2/neu-) cells (A); and SKOVip1 (Her 2/neu+) cells (B).

FIG. 12. Graphs showing cytotoxicity of Herceptin-PEG-PG-TXL in MDA 435/neo cells (A) and MDA 435/e B2 cells (B).

FIG. 13. Graph showing cytotoxicity of C225-PEG-PG-Adr in A431 Cells: 6 hours exposure followed by washing, and additional 72 hours incubation.

### DESCRIPTION OF THE INVENTION

The present invention is directed to novel conjugates useful for the selective delivery of therapeutic agents (e.g., chemotherapeutic drugs, hormonal agents and diagnostic agents) and other compounds and agents to tumors or another target tissue. The invention is also directed to novel methods of synthesizing and using such conjugates. Preferred embodiments of the invention comprise a ligand, such as a monoclonal antibody (e.g., C225 or Herceptin), indirectly coupled to a therapeutic agent, such as a chemotherapeutic drug. The coupling is achieved by conjugating the ligand site-specifically to the termini of a polymer-therapeutic agent conjugate using a polymer spacer or linker (e.g., a PEG spacer).

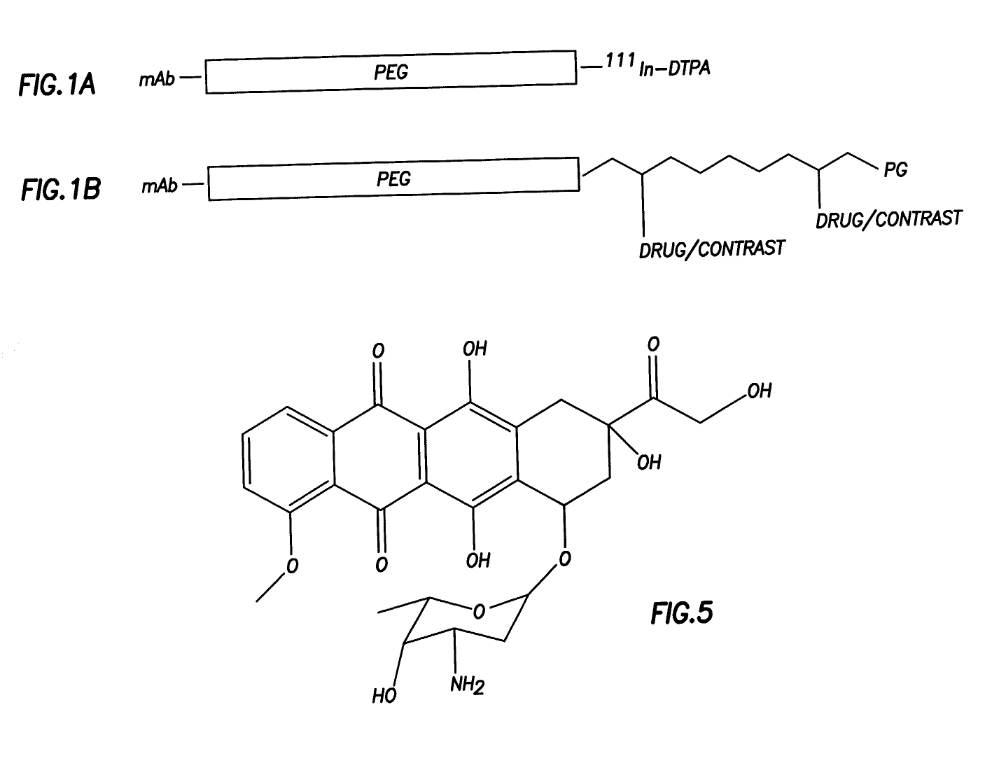

Copending provisional patent application 60/286,453, incorporated herein by reference in its entirety, describes the coupling of a radionuclide ¹¹¹In to a terminus of polyethylene glycol (PEG) chain which was in turn attached to C225, a mAb directed against EGF receptor. Specifically, as shown in FIG. 1A, a polyethylene glycol (PEG) conjugated monoclonal antibody (mAb) with a radionuclide attached to one terminus of the PEG chain and the antibody to the another terminus of PEG chain was designed and synthesized (See also, X-X. Wen et al, Poly(ethylene glycol) conjugated anti-EGF receptor antibody C225 with radiometal chelator attached to the termini of polymer chains. Bioconjug. Chem. 12:545-553, 2001). The conjugate exhibited significantly reduced nonspecific interaction and improved nuclear imaging property (X-X. Wen et al, Improved imaging of ¹¹¹In-DTPA-poly(ethylene glycol) conjugated anti-EGF receptor antibody C225. J. Nucl. Med., 42:1530-1537, 2001).

It has been discovered that conjugation of a receptor-homing ligand to the end of a polymer chain through a PEG linker enhances the targeted delivery of therapeutic agents. As shown in the Examples, mAbs were coupled site-specifically to the termini of PG-drug conjugates via a PEG linker. Specifically, C225 (an anti-EGF receptor mAb), and Herceptin (an anti-Her2/neu mAb), were site-specifically conjugated to the termini of poly(1-glutamic acid)-drug conjugates through a PEG spacer. A schematic of the construct is shown in FIG. 1B.

The novel conjugates of the invention demonstrated enhanced cellular uptake of the polymeric construct into tumor cells overexpressing EGF receptors and for Her2/neu receptors. The polymeric immunoconjugates maintained the binding affinity of the corresponding mAbs. Specifically, C225 and Herceptin conjugates bound to target cell surfaces. In addition, the C225 conjugate appeared to be internalized. As shown in the biologic assays, the attachment of drugs to the polymeric carrier through hydrolytically stable amide linkage and the efficient cellular internalization yielded significantly increased selective cytotoxicity against target cells. Further, targetable polymeric nanoparticles formed when Adriamycin was used as the drug to conjugate to mAb-PEG-PG carrier.

Accordingly, one embodiment of the invention is directed to a conjugate molecule comprising: a ligand; a polymer spacer; a polymer carrier; and a therapeutic agent. The conjugate molecule is useful for the selective delivery of the therapeutic agent to tumors or other tissues with biological receptors. Preferably, the ligand is bonded to the polymer spacer, the polymer spacer is bonded to the polymer carrier, and the polymer carrier is bonded to the therapeutic agent. As used herein, “bonded” refers to any physical or chemical attachment, including, but not limited to, covalent bonding, ionic or chelating interactions.

More preferably, the ligand is bonded to the polymer spacer via a covalent bond, the polymer spacer is bonded to the polymer carrier via a covalent bond, and the polymer carrier is bonded to the therapeutic agent directly via a covalent bond, or indirectly using a linker. For example, the ligand and polymer spacer may be joined by an amide bond, a thioether (S-C) bond, a disulfide (S-S) bond, or a thiourethane bond, more preferably, an amide, thioether or disulfide bond, and, most preferably, a thioether bond. The polymer spacer and polymer carrier may be joined, for example, by an amide bond, a thioether (S-C) bond, a disulfide (S-S) bond, a thiourethane bond, a carbonate bond or a urethane bond, more preferably, an amide or a urethane bond, and, most preferably, an amide bond. The polymer carrier and therapeutic agent may be bonded to each other, for example, by an amide, thioether, disulfide, thiourethane, hydrazone or ester bond, and more preferably, by an amide or ester bond. Alternately, the polymer carrier and therapeutic agent can be bonded or joined using a linker. Useful linkers include, but are not limited to, aliphatic chains, lipids, amino acids or peptides. In the latter embodiments, the polymer carrier is preferably covalently bonded to the linker, and the linker is preferably covalently bonded to the therapeutic agent.

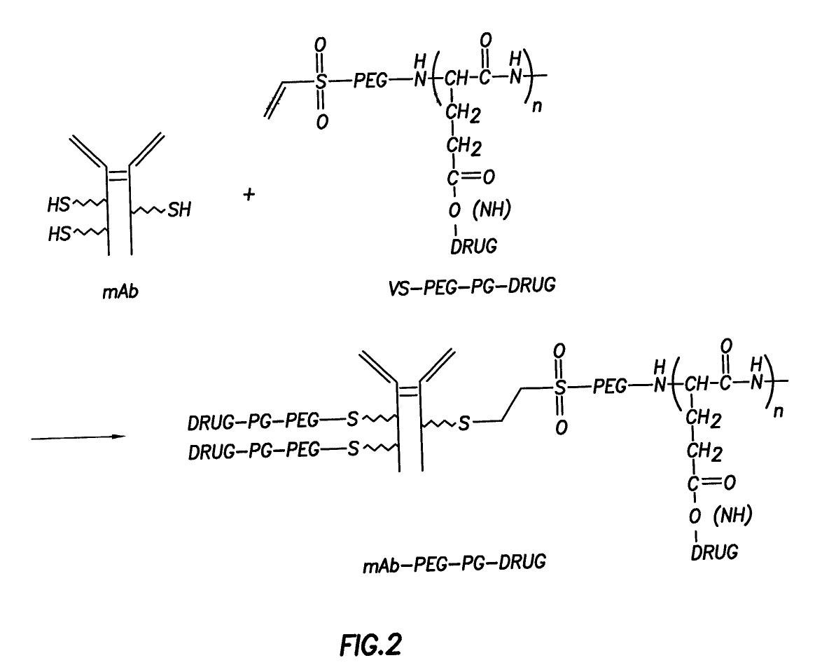

Further, as shown in FIG. 2, more than one polymer spacer-polymer carrier-therapeutic agent construct may be bonded to a single ligand or antibody. Multiple therapeutic agents may be bonded to the polymer carrier. Ligands different from those attached to the PEG chain terminus may be bonded to the side chains of the polymer carrier.

The polymer carrier to which the therapeutic agent or other compound is attached is preferably poly(1-glutamic acid). However, other polymers, particularly those which are biocompatible, water-soluble, biodegradable, and have multiple side-chain functional groups that allow attachment of multiple drug molecules, may be used without departing from the scope of the invention. These polymers include, but are not limited to, poly(d-glutamic acid), poly(d1-glutamic acid), poly(1-aspartic acid), poly(d-aspartic acid), poly(d1-aspartic acid), polylysine, polysaccharides, polyhydroxypropylmethacryamide (HPMA), dextran, poly(hydroxypropylglutamine), poly(hydroxyethylglutamine), hyaluronic acid, carboxymethyl dextran, polyacrylic acid and chitosan, and copolymers between two or more of them.

The polymer carrier can generally have any number average molecular weight, and preferably has a number average molecular weight of at least about 1,000 daltons. The poly(1-glutamic acid) preferably has a number average molecular weight of about 1,000 daltons to about 100,000 daltons. The other polymers listed above as carriers preferably have a number average molecular weight of about 1,000 daltons to about 150,000 daltons.

The polymer spacer between the ligand and the polymer is preferably PEG. However, other linear polymers, particularly those which are biocompatible and uncharged, may be used without departing from the scope of the invention. These polymers include, but are not limited to, a polyamino acid, such as polyglycine, polytyrosine, polyphenylalanine, dextran, polysaccharides, polypropylene oxide (PPO), a copolymer of polyethylene glycol (PEG) with PPO, polyglycolic acid, polyvinyl pyrolidone, polylactic acid and polyvinyl alcohol.

The polymer spacer can generally have any number average molecular weight, and preferably has a number average molecular weight of at least about 1,000 daltons. The polyethylene glycol preferably has a number average molecular weight of about 1,000 daltons to about 100,000 daltons. The other polymers listed above as spacers preferably have a number average molecular weight of about 1,000 daltons to about 100,000 daltons.

The ligand (or targeting moiety) can generally be any ligand, and preferably is an antibody or its fragments, a peptide or a protein. The antibody can generally be a monoclonal antibody, or a polyclonal antibody. For example, useful antibodies include, but are not limited to, C225, Herceptin, Rituxan, phage library antibodies, anti-CD, DC101, antibodies to the integrins alpha v-beta 3 (such as LM609), antibodies to VEGF receptors, antibodies to VEGF, or any other suitable antibody. The antibody can be an antibody fragment such as F(ab′)₂, Fab′, or ScFv fragment or an antibody fragment such as chimeric (c) 7E3Fab (c7E3Fab) that binds to integrin receptors. The antibody can be a humanized antibody. The peptide can generally be any peptide, such as a cell surface targeting peptide, and preferably is a growth factor, such as VEGF (Vascular Endothelial Growth Factor)-A, -B, -C or -D, PDGF (Platelet-Derived Growth Factor), Angiopoietin-1 or -2, HGF (Hepatocyte Growth Factor), EGF (Epidermal Growth Factor), bFGF (Basic Fibroblast Growth Factor), cyclic CTTHWGFTLC, cyclic CNGRC, or cyclic RGD-4C. The protein can generally be any protein, such as annexin V, interferons (e.g., interferon α, interferon β), tumor necrosis factors, endostatin, angiostatin, or thrombospondin, and preferably is annexin V, endostatin, angiostatin, interferon-α or interferon-β. More preferably, the ligand is a monoclonal antibody, such as a C225, Herceptin or c7E3Fab antibody, or a protein, such as annexin V. Preferably, the ligand has affinity for a target tissue. Preferred ligands bind specifically to receptors or other binding partners on the target tissue.

As used herein “therapeutic agent” broadly includes, but is not limited to, drugs, chemotherapeutic drugs/agents, diagnostic agents, hormonal drugs/agents, and other compounds and compositions useful in the treatment, diagnosis and monitoring of disease. The invention is particularly useful for the delivery of chemotherapeutic agents. Chemotherapeutic agents useful in the practice of the invention include, but are not limited to, Adriamycin (Adr or doxorubicin), daunorubicin, paclitaxel (Taxol), docetaxel (taxotere), epothilone, camptothecin, cisplatin, carboplatin, etoposide, tenoposide, geldanamycin, methotrexate, maytansinoid DM1 or 5-FU. Preferably, the chemotherapeutic agent is Adriamycin or paclitaxel, and, more preferably, is Adriamycin. Other therapeutic agents that can be used include, but are not limited to, magnetic resonance imaging contrast agents such as gadolinium-DTPA (Gd-DTPA), and near-infrared optical imaging agents such as Cy 5.5, indocyanine green (ICG) and its derivatives, and Alexa fluor. However, the invention is not limited to the foregoing, and other compounds and agents can be used without departing from the scope of the invention.

Another embodiment of the invention is directed to a composition comprising a plurality of nanoparticles. The nanoparticles comprise a plurality of the conjugate molecules described herein. Preferably, the therapeutic agent in the nanoparticles is Adriamycin. In this embodiment, the polymer spacer and polymer carrier have hydrophilic/hydrophobic characters or hydrophobic/hydrophilic characters. For example, as shown in Example 2, the PEG block in PEG-PG-Adr is hydrophilic, and the PG-Adr block in the copolymer is hydrophobic.

Still another embodiment is directed to compositions comprising any of the conjugate molecules described herein and a pharmaceutically acceptable carrier. As used herein, “pharmaceutically acceptable carrier” includes any and all solvents, dispersion media, coatings, antibacterial and antifungal agents and isotonic agents and the like. The use of such media and agents for pharmaceutically active substances is well known in the art. For example, the carrier may comprise water, alcohol, saccharides, polysaccharides, drugs, sorbitol, stabilizers, colorants, antioxidants, buffers, or other materials commonly used in pharmaceutical compositions. Except insofar as any conventional media or agent is incompatible with the active ingredient, its use in the therapeutic compositions is contemplated. Supplementary active ingredients can also be incorporated into the compositions.

The phrase “pharmaceutically acceptable” also refers to molecular entities and compositions that do not produce an allergic or similar untoward reaction when administered to an animal or a human.

A preferred composition is a pharmaceutical preparation suitable for injectable use. Pharmaceutical preparations of the invention suitable for injectable use include sterile aqueous solutions or dispersions and sterile powders for the preparation of sterile injectable solutions or dispersions. Preferably, the preparations are stable under the conditions of manufacture and storage and are preserved against the contaminating action of microorganisms, such as bacteria and fungi. The carrier may be a solvent or dispersion medium containing, for example, water, ethanol, polyol (for example, glycerol, propylene glycol, and liquid polyethylene glycol, and the like), suitable mixtures thereof, and vegetable oils. The prevention of the action of microorganisms may be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

Sterile injectable solutions may be prepared by incorporating the active compounds in the required amount in the appropriate solvent with various of the other ingredients enumerated above, as required, followed by filtered sterilization. Generally, dispersions may be prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation include vacuum-drying and freeze-drying techniques which yield a powder of the active ingredient plus any additional desired ingredient from a previously sterile-filtered solution thereof.

For parenteral administration in an aqueous solution, the solution is preferably suitably buffered, if necessary, and the liquid diluent first rendered isotonic with sufficient saline or glucose. These particular aqueous solutions are especially suitable for intravenous and intraperitoneal administration.

A further embodiment of the invention is directed towards methods for selectively targeting tumors or other target tissues with biological receptors using any of the herein described conjugate molecules and compositions. For example, one such embodiment is directed to a method for selectively delivering a therapeutic agent to a target tissue in a patient comprising: administering a conjugate molecule to a patient having the target tissue, wherein the conjugate molecule comprises: a ligand with affinity for the target tissue; a polymer spacer; a polymer carrier; and a therapeutic agent. Preferably, the ligand is bonded to the polymer spacer, the polymer spacer is bonded to the polymer carrier, and the polymer carrier is bonded to the therapeutic agent. Preferably, the ligand is an antibody, the polymer spacer is polyethylene glycol, and the polymer carrier is poly(1-glutamic acid).

Because of the affinity of the ligand for the target tissue, the therapeutic agent is selectively delivered to the tissue, where it exerts its therapeutic effect. For example, in a preferred embodiment, the therapeutic agent is a cytotoxic agent which exerts a cytotoxic effect on the target tissue.

The administering step may be performed parenterally, e.g., by intravascular, intraperitoneal, intramuscular or intratumoral injection. The conjugate molecule may be administered by inhalation or another suitable route. Preferably, administration is by intravascular injection.

The target tissue may be any desired tissue, including, but not limited to, a tumor or other neoplasm, inflammatory, infectious, reparative or regenerative tissue (including post trauma and post surgery tissues). As used herein, “tumor” includes benign and malignant tumors or neoplasia. In one embodiment, the target tissue is a solid tumor, such as breast cancer, ovarian cancer, colon cancer, lung cancer, head and neck cancer, a brain tumor, liver cancer, a pancreatic tumor, bone cancer, or prostate cancer. Alternately, the target tumor may be a malignancy such as leukemia or lymphoma. The patient can be any animal. Preferably the patient is a mammal. The mammal can be a human, a dog, a cat, a horse, a cow, a pig, a rat, a mouse or other mammal. More preferably, the patient is a human. As used herein, “patient” broadly includes, but is not limited to, a human or any animal being treated, tested or monitored in any kind of therapeutic, diagnostic, research, development or other application.

Additional embodiments of the invention are directed towards other therapeutic applications using the herein described conjugate molecules. One such embodiment is directed to a method of treating a patient having or suspected of having a diseased tissue, the method comprising administering a therapeutically effective amount of a conjugate molecule to the patient, wherein the conjugate molecule comprises: a ligand with affinity for the diseased tissue; a polymer spacer; a polymer carrier; and a therapeutic agent. Preferably, the ligand is bonded to the polymer spacer, the polymer spacer is bonded to the polymer carrier, and the polymer carrier is bonded to the therapeutic agent. Any of the conjugates described herein can be used.

Because of the affinity of the ligand for the diseased tissue, the therapeutic agent is selectively delivered to the tissue, where it exerts its therapeutic effect. For example, when the therapeutic agent is a chemotherapeutic or cytotoxic agent and the diseased tissue is a tumor, the therapeutic effect may include inhibition or killing of the tumor cells.

The administering step may be performed parenterally, e.g., by intravascular, intraperitoneal, intramuscular or intratumoral injection. The conjugate molecule may be administered by inhalation or another suitable route. Preferably, administration is by intravascular injection. The dosage of the conjugate molecule can be increased or decreased to modulate the therapeutic effect on the targeted diseased tissue.

The patient can generally be any animal. Preferably the patient is a mammal. The mammal can be a human, a dog, a cat, a horse, a cow, a pig, a rat, a mouse or other mammal. More preferably, the patient is a human. The diseased tissue may be any type of tissue, including, but not limited to, a tumor or other neoplasm, inflammatory, infectious, reparative or regenerative tissue. In one embodiment, the diseased tissue is a tumor, and, more preferably, is a solid tumor such as breast cancer, ovarian cancer, colon cancer, lung cancer, head and neck cancer, a brain tumor, liver cancer, a pancreatic tumor, bone cancer, or prostate cancer. Alternately, the target tumor may be a malignancy such as leukemia or lymphoma.

As used herein the term “treating” a tumor is understood as including any medical management of a subject having a tumor. The term would encompass any inhibition of tumor growth or metastasis, or any attempt to visualize, inhibit, slow or abrogate tumor growth or metastasis. The method includes killing a cancer cell by non-apoptotic as well as apoptotic mechanisms of cell death.

In the foregoing methods, a therapeutically effective amount of the conjugate molecules of the invention is preferably administered to achieve the desired effect. The actual dosage amount of a composition comprising the conjugate molecule of the present invention administered to the patient to achieve the desired effect (e.g., delivery to or treatment of the target or diseased tissue) can be determined by physical and physiological factors such as body weight, severity of condition, the type of disease being treated, previous or concurrent therapeutic interventions, idiopathy of the patient and route of administration, as well as other factors known to those of skill in the art. The practitioner responsible for administration will, in any event, determine the concentration of active ingredient(s) in a composition and appropriate dose(s) for the individual subject.

The invention also includes methods for synthesizing the novel conjugates and compositions of the invention. One such method for synthesizing a conjugate molecule comprising a therapeutic agent and ligand comprises the steps of: providing a polymer spacer-polymer carrier construct having a sulfhydryl-reactive vinyl sulfone group at one end of the polymer spacer; conjugating the therapeutic agent to the polymer carrier to form a vinyl sulfone-polymer spacer-polymer carrier-therapeutic agent construct; pretreating the ligand to introduce a sulfhydryl group on the ligand; and combining the ligand with the vinyl sulfone-polymer spacer-polymer carrier-therapeutic agent construct, wherein the vinyl sulfone group reacts with the sulfhydryl group to form a conjugate molecule comprising the ligand, the polymer spacer, the polymer carrier, and the therapeutic agent, and wherein the ligand is bonded to the polymer spacer, the polymer spacer is bonded to the polymer carrier, and the polymer carrier is bonded to the therapeutic agent.

Another method for synthesizing a conjugate molecule of the invention comprises the steps of: introducing at least one protected sulfhydryl group (SH) to an end of a polymer spacer; conjugating the polymer spacer to a polymer carrier to form a protected SH-polymer spacer-polymer carrier construct; conjugating a therapeutic agent to the polymer carrier to form a protected SH-polymer spacer-polymer carrier-therapeutic agent construct; pretreating a ligand to introduce a sulfhydryl reactive functional group on said ligand; deprotecting the protected SH group to obtain a free SH group; and combining the pretreated ligand with the SH-polymer spacer-polymer carrier-therapeutic agent construct, wherein the SH group reacts with the sulfhydryl reactive functional group to form a conjugate molecule comprising the ligand, the polymer spacer, the polymer carrier, and the therapeutic agent. In the resulting conjugate, the ligand is bonded to the polymer spacer, the polymer spacer is bonded to the polymer carrier, and the polymer carrier is bonded to the therapeutic agent.

In this method, the ligand is preferably pretreated with a suitable agent, such as vinyl sulfone or maleimide to introduce the sulfhydryl reactive functional group. Preferably, the SH group is deprotected to obtain a free SH group before combining with the ligand.

Still another method for synthesizing a conjugate molecule comprises the steps of: providing a polymer-spacer-polymer carrier-therapeutic agent construct; introducing a protected amine to an end of the polymer spacer to form a protected amine-polymer spacer-polymer carrier-therapeutic agent construct; deprotecting the protected amine-polymer spacer-polymer carrier-therapeutic agent construct to obtain a free amine-polymer spacer-polymer carrier-therapeutic agent construct; and combining the free amine-polymer spacer-polymer carrier-therapeutic agent construct with a ligand having a carboxylic acid group. The carboxylic acid in the ligand conjugates with the free amine to form an amide bond, thereby forming a conjugate molecule comprising the ligand, the polymer spacer, the polymer carrier and the therapeutic agent. In the resulting conjugate molecule, the ligand is bonded to the polymer spacer, the polymer spacer is bonded to the polymer carrier, and the polymer carrier is bonded to the therapeutic agent.

Ligands, polymer spacers, polymer carriers and therapeutic agents useful in the practice of the foregoing synthetic methods include, but are not limited to, any of the ligands, polymer spacers, polymer carriers and therapeutic agents disclosed herein. For example, the therapeutic agent may comprise a contrast agent or a chemotherapeutic drug. In the resulting conjugates, the ligand is preferably bonded to the polymer spacer via a covalent bond, the polymer spacer is bonded to the polymer carrier via a covalent bond, and the polymer carrier is bonded to the therapeutic agent directly via a covalent bond or indirectly using a linker.

The use of the above described conjugate molecules is advantageous over those previously described in the art. Preferred embodiments of the conjugate molecules are useful for the targeted treatment of tumors and other diseased tissue. Preferred embodiments have improved in vivo half lives and exhibit reduced or eliminated accumulation in the liver. The use of polymers reduces non-specific interaction with non-target tissues and reduces background activity. Attachment of the therapeutic agent and polymer carrier to the ligand with a polymer spacer instead of to the ligand directly improves retention of the ligand's receptor binding affinity. The conjugate molecule design strategy is flexible, and allows for the preparation of a wide array of molecules for different diagnostic and clinical uses. It allows both passive targeting (when ligand is not attached) and active targeting (when ligand is attached).

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Materials and Methods

a. Materials

Diisopropylcarbodiimide (DIC), dimethylformamide (DMF), poly(1-glutamic acid) (PG, MW 31K), p-nitrophenol, p-nitrophenyl chloroformate (PNP), dimehtylaminopyridine (DMAP), 2-isobutoxy-1-isobutoxycarbonyl-1,2-dihydroquinoline) (IIDQ) were purchased from Sigma-Aldrich (Milwaukee, Wis.). Paclitaxel and Adriamycin-hydrochloride (Adr·HCl) were obtained from Hande Tech. (Houston, Tex.). BODIPY FL hydrazide dye was obtained from Molecular Probes (Eugene, Oreg.). Vinylsulfonyl N-hydroxysuccinimidyl PEG (VS-PEG-NHS, MW 3400) and NH₂-PEG-OH were obtained from Shearwater (Huntsville, Ala.). N-succinimidyl S-acetylthioacetate (SATA), γ-maleimidobutyric acid N-hydroxysuccinimide ester (GMBS), N-succinimidyl 3-[2-pyridyldithio]propionate (SPDP), dithiothreitol (DTT), and hydroxyamine were obtained from Pierce Chemical Co. (Rockford, Ill.). C225 is a human-mouse chimeric monoclonal antibody that targets epidermal growth factor receptor (EGFR or EGF receptor) and was kindly provided by ImClone Systems Inc. (New York, N.Y.). Herceptin (Trastuzumab) was obtained from Genentech (San Francisco, Calif.).

UV measurements were recorded using a Beckman DU640 spectrophotometer (Fullerton, Calif.). ¹H-NMR spectra were obtained with a Bruker 300 MHz instrument (Billerica, Mass.).

b. Gel Permeation Chromatography (GPC)

GPC was performed with a Waters HPLC system (Waters Corporation, Milford, Mass.) consisting of a 717 plus autosampler, a 2410 refractive index detector, and a 2487 dual λ UV detector. Samples were eluted with 0.1 M phosphate buffer (pH 7.4) containing 0.1% LiBr at a flow rate of 1 ml/minute through a Superdex 200 column (Amersham Pharmacia Biotech, Piscataway, N.J.) (system 1). Alternatively, the mobile phase was run at a rate of 0.5 ml/minute through the same column (system 2).

c. Ion-Exchange Chromatography

The system consisted of an AKTA fast protein liquid chromatography (FPLC) (Amersham Pharmacia Biotech) and a Resource Q anion exchange column (Amersham Pharmacia). The mobile phase was run from Buffer A (20 mM Tris buffer, pH 7.5) to Buffer B (20 mM Tris buffer containing 0.15 or 1.0 N NaCl, pH 7.5) in a linear fashion at a flow rate of 3 ml/minute for 20 ml (6.67 minutes). The column was eluted with 100% Buffer B for the rest of the chromatographic period.

d. Analytical Assays Used to Determine the Degree of mAb Modification

The degree of substitution of C225 by SATA was estimated by measuring the changes in the concentrations of free amino groups using 2,4,6-trinitrobenzenesulfonic acid (TNBS) assay, and by monitoring the presence of sulfhydryl groups using Ellman's test (GT Hermanson, ed., Amine detection reagents. Bioconjugate Techniques. San Diego, Academic Press. pp. 112-114 and pp. 88-90, 1996).

e. Determination of the Molar Ratio of Components in Immunoconjugates

The concentration of each component of the conjugate was determined and the molar ratio was calculated. The concentration of the antibody was measured by UV at 650 nm using the Bio-Rad Laboratory protein assay kits (Hucoles, Calif.). In these measurements, known concentration of C225 or Herceptin was used as a reference standard and PEG-PG-BODIPY FL was used as the background. Taxol, Adr, and BODIPY FL concentrations were quantified by determining the absorbance at 230 nm, 480 nm, or 503 nm. The concentration of paclitaxel in Her-PEG-PG-TXL or C225-PEG-PG-TXL was further determined by a hydrolysis/HPLC method. Alternatively, the concentration of TXL in the immunoconjugates was estimated by assuming a molar ratio of 1:1 between mAb and PEG-PG-TXL. The assumption is based on UV absorbance of mAb-PEG-PG-BODIPY FL where the concentration of BODIPY FL, and hence the molar ratio between mAb and PEG-PG, could be conveniently determined by UV measurements.

f. Quantification of TXL concentration in mAb-PEG-PG-TXL by Hydrolysis and HPLC Analysis

Ten mg of PEG-PG-TXL dissolved in 200 μl of 1 N NaHCO₃, or 1.0 ml of mAb-PEG-PG-TXL solution containing 50 mg of NaHCO₃ with known concentration of mAb was charged into a 5-ml vial with a septum, a needle for gas release, and a stirring bar. Into the vial was added 600 μl of a 50% H₂O₂ solution and 1 ml of CH₂Cl₂. The mixture was stirred vigorously overnight at room temperature. The aqueous portion was extracted with methylene chloride twice and the organic portions were combined. Taxol concentration was obtained from HPLC analysis with the following conditions: 1 mL/minute flow rate, gradient of water/CH₃CN (changing acetonitrile from 0% to 40%), column: Nova Pak (3.9×150 mm) and UV detector at 228 nm. A standard curve was constructed using a range of Taxol solution in methylene chloride with concentrations ranging between 0.5 and 6 mg/ml. Ten μl aliquot was injected from each standard and each extracted solution.

### Example 2

### Synthesis of Conjugates

To conjugate homing ligand to the end of a PG chain through a PEG spacer, a linear PEG-PG conjugate that contains a sulfhydryl reactive vinyl sulfone (VS) group at the end of the PEG block of the copolymer was synthesized. The anticancer agent Adriamycin (Adr) or paclitaxel (Taxol, TXL) was conjugated to the side chain carboxyl groups in the PG block of VS-PEG-PG via p-nitrophenol activated esters, IIDQ, or carbodiimide-mediated reaction. Subsequent coupling of mAb, which was pre-treated with N-succinimidyl S-acetylthioacetate (SATA) and hydroxyamine to introduce sulfhydryl groups, yielded the final conjugates mAb-PEG-PG-drug. The synthetic scheme is shown in FIG. 2. The mAb used in this study included C225, a mAb directed against epidermal growth factor receptor (EGFR) and Herceptin, a mAb directed against Her-2/neu receptor. Both receptors are overexpressed in a variety of solid tumors. For example, EGFR is overexpressed on the cells of over one-third of all solid tumors, including bladder, breast, colon, ovarian, prostate, renal cell, squamous cell, non-small cell lung, and head and neck carcinomas.

a. Synthesis of VS-PEG-PG

Into 500 mg of PG in 1 M phosphate buffer (pH=8) was added 200 mg of VS-PEG-NHS in five fractions in a course of 2 hours. The reaction mixture was stirred for an additional 5 hours at room temperature. Ninhydrin spray was used to monitor the consumption of unreacted NH₂ at the terminal of PG polymer. To stop the reaction, the reaction mixture was acidified with 1 N HCl to pH 3.0 and the precipitate was recovered by centrifugation at 3,000 rpm for 10 minutes. The solid was washed two times with distilled water to remove free PEG and lyophilized to yield 360 mg of the conjugate product in acid form.

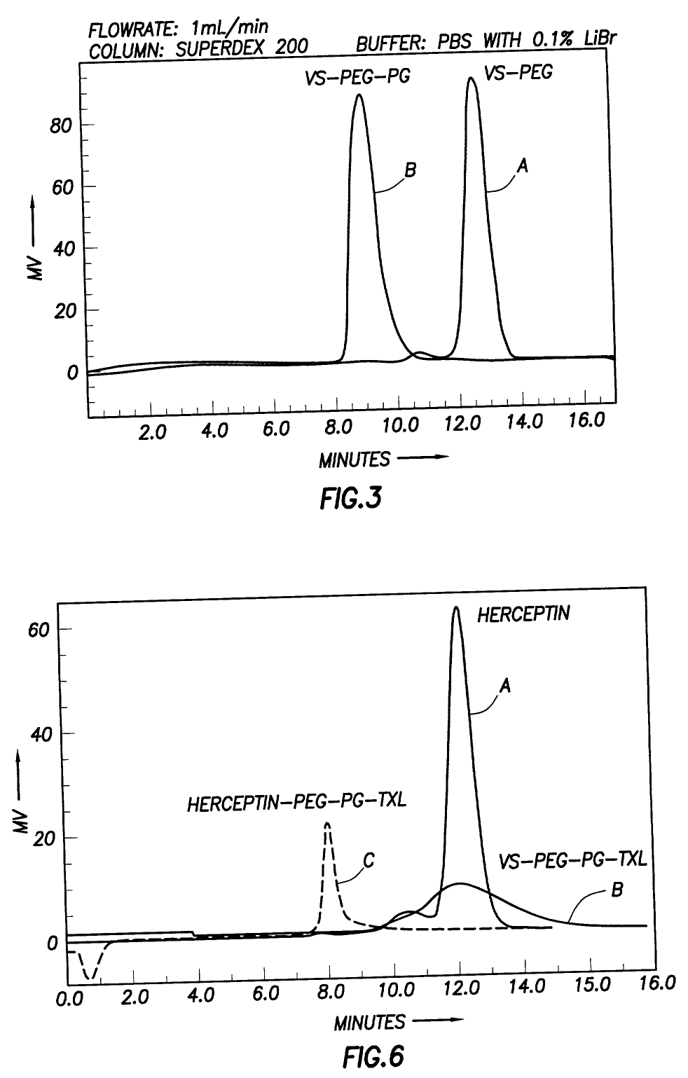

The simple purification scheme removed most of the unreacted PEG as revealed by GPC analysis (system 1) (FIG. 3). Specifically, FIG. 3 is a comparison of GPC chromatograms of VS-PEG-PG conjugate (B) and VS-PEG (A). The concentrations of the compounds were monitored by RI detector. (Conditions: Flow Rate—1 mL/minute; Column—Superdex 200; Buffer—PBS with 0.1% LiBr).

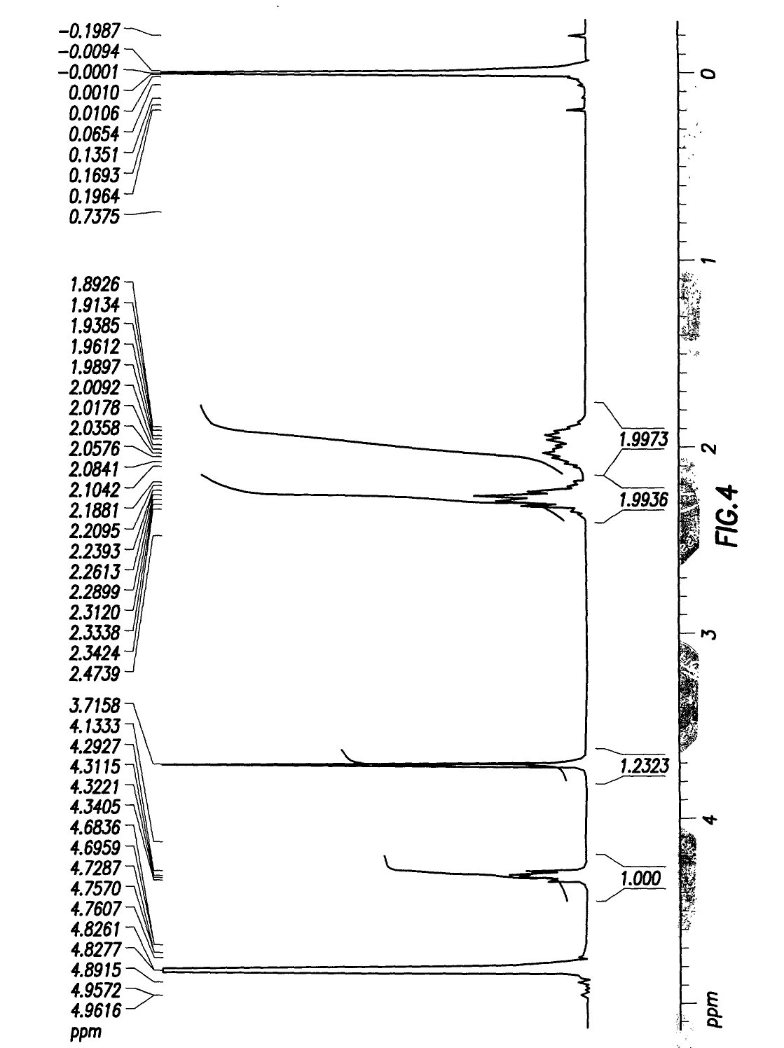

Since both unconjugated PG and VS-PEG-PG had the same retention times of 9.0 minutes, further study was performed to verify that the isolated product was indeed VS-PEG-PG conjugate. An ¹H NMR spectrum of the isolated product, VS-PEG-PG, is shown in FIG. 4. The spectrum revealed the presence of characteristic peaks attributable to both PEG (δ3.72 ppm, s) and PG (δ4.29-4.34, 2.19-2.34 ppm, 1.89-2.04 ppm for α-CH, γ-CH₂, and β-CH₂, respectively). Furthermore, the molar ratio between PEG and PG was 0.96 based on the integrals between CH₂ of PEG and α-CH of PG (FIG. 4). These data confirmed that the peak at 9.0 minutes in GPC chromatogram of the isolated product is attributed to VS-PEG-PG rather than that of the free PG.

b. Synthesis of VS-PEG-PG-TXL and VS-PEG-PG-Adr

Into a solution of 250 mg VS-PEG-PG in 10 ml DMF was dissolved 150 mg (176 μmol) of paclitaxel, 30 mg of DIC (238 μmol), 75 μL of pyridine and a trace amount of DMAP. The reaction mixture was stirred overnight at room temperature. After evaporation of the solvent under vacuum, the residual was dissolved in 0.1N NaHCO₃. The aqueous solution was filtered through a 0.22-μm filter and dialyzed against distilled water overnight using membrane with molecular weight cut-off (MWCO) of 10K (Spectrum Laboratories, Rancho Dominguez, Calif.). The product was recovered as lyophilized powder. Yield: 379 mg polymer conjugate. Paclitaxel content: 21.6% (w/w) based on UV measurement at 230 nm. Each polymer chain contained about 11 TXL molecules. TXL yield: 54.6%. No free paclitaxel was detected by silica gel thin layer chromatography (MeCl₂/methanol, 4/1, v/v) and by GPC (system 1).

Adr was conjugated to VS-PEG-PG via the DIC-mediated coupling reaction using similar procedures. (The structure of Adr is shown in FIG. 5; the drug was conjugated to VS-PEG-PG polymer through its amino groups on the sugar moiety.) Thus, into a solution of 100 mg VS-PEG-PG in 5 ml DMF was added Adr free amine (40 mg, 74 μmol), 30 μl DIC (24.3 mg, 192 μmol), 100 μl pyridine, and trace amount of DMAP. Adr free amine was obtained by extracting an aqueous solution of Adr·HCl and triethylamine (molar ratio 1:3) with chloroform. The reaction mixture was worked up as follows: The aqueous solution of polymer conjugate was acidified with 1.0 HCl. The precipitate was collected by centrifugation, washed with water, re-dissolved in 0.1 N NaHCO₃, and dialyzed. GPC (system 2) revealed the absence of free Adr in the isolated product. The amount of Adr in the polymer was estimated to be 15% (w/w) as measured by UV at 480 nm. Each polymer chain contained about 11 Adr molecules. Yield: 120 mg polymer conjugate, Yield of Adr, 45%.

The fluorescent dye BODIPY was conjugated to VS-PEG-PG to facilitate confocal fluorescent microscopic study. Briefly, 5 mg BODIPY-hydrazide (16.3 μmol) was conjugated to 120 mg of VS-PEG-PG to yield 150 mg of VS-PEG-PG sodium salt. Approximately 5 dye molecules were attached to each polymer chain.

c. Synthesis of Herceptin-PEG-PG-TXL, Herceptin-PEG-PG-BODIPY, and C225-PEG-PG-Adr

Into a solution of 50 mg C225 or Herceptin (0.33 μmol) in 5 ml PBS (pH=7.2) was added an aliquot of SATA in DMF (190 μl, 8 mg/ml, molar ratio: 1:20). After being stirred for 1 hour at room temperature, 0.5 ml of 50 M hydroxylamine aqueous solution was added into the solution. The reaction mixture was stirred for an additional 2 hours, then concentrated to 1-2 ml by ultracentrifugation (MWCO, 10K; Millipore Corp., Bedford, Mass.). The resulting SH-containing mAb was purified with a PD-10 column to remove small molecular weight contaminants. Finally, mAb was mixed with VS-PEG-PG-TXL, VS-PEG-PG-Adr, or VS-PEG-PG-BODIPY with a molar ratio of mAb to polymer of 1:8-1:10. After being stirred at 4° C. overnight, the solution was passed through a nickel affinity column (FreeZyme conjugate purification kit, Pierce Chemical Co., Rockford, Ill.) to remove unreacted polymer, followed by purification with an anion exchange chromatography to remove free mAb from polymer bound mAb. The yield of mAb was calculated to be 8-10%. The molar ratios of Herceptin to PEG-PG polymer and C225 to PEG-PG were 1 based on the measurements of protein and BODIPY FL concentrations. Using the ratio of Herceptin to PEG-PG of 1, the calculated TXL content in the conjugate was 4.3%. TXL content obtained from hydrolysis/HPLC assay was 6.65%, which suggests a molar ratio of Herceptin to PEG-PG of 1.8. Thus, the molar ratios of mAb to PEG-PG in immunoconjugates varied between 1.0 to 1.8.

When GPC Superdex 200 chromatography was applied to the affinity purified and ion-exchange purified Her-PEG-PG-TXL conjugate, a single peak at 8.15 minutes was found Specifically, FIG. 6 shows the GPC elution profile of Herceptin (A), VS-PEG-PG-TXL (B), and purified Herceptin-PEG-PG-TXL conjugate (C) using a Superdex 200 column (2.4×20 cm). The concentrations of the compounds were monitored by RI detector. Herceptin and PEG-PG-TXL appeared almost in the same position (retention time 12.12-12.39 minutes) (FIG. 6), although their molecular weights are approximately 150,000 and 41,000, suggesting that the hydrodynamic volume of PEG-PG-TXL is similar to that of the globular protein IgG.

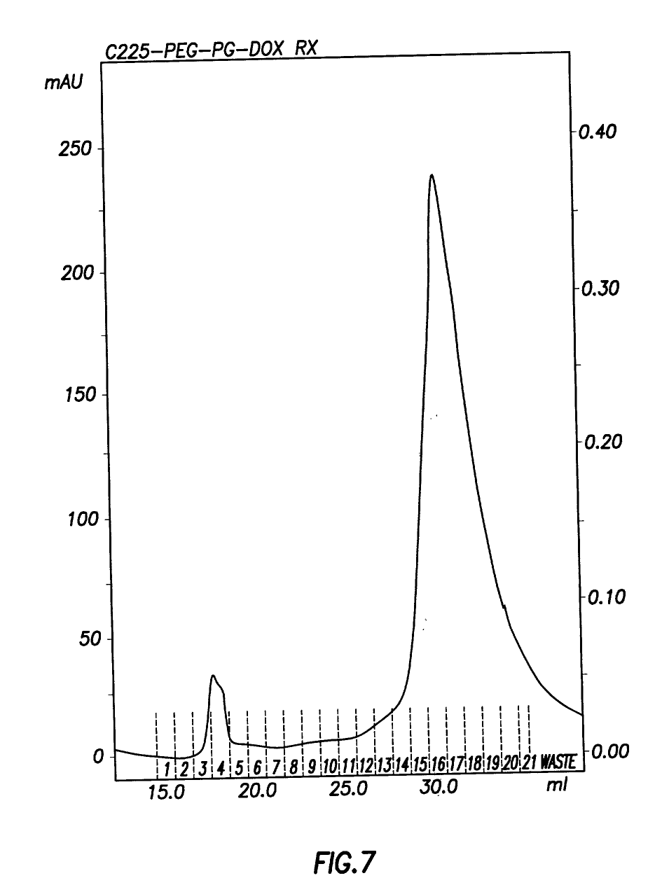

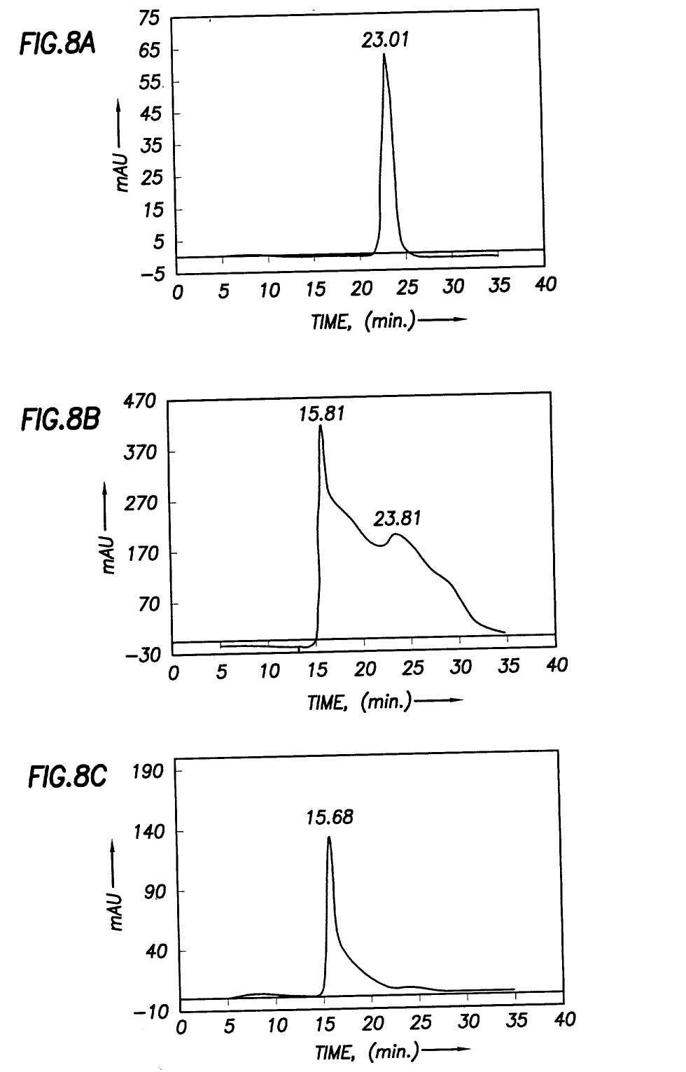

C225-PEG-PG-Adr was purified following a similar protocol. After removal of unconjugated PEG-PG-Adr polymer from the C225-PEG-PG-Adr conjugate by affinity chromatography, the immunoconjugate was further purified by anion exchange chromatography to remove unconjugated C225. Specifically, FIG. 7 shows the purification of C225-PEG-PG-Adr by FPLC using a Resource Q anion-exchange column. Each fraction was 0.5 ml. From the FPLC elution profile, the fractions (fractions 3-5) corresponding to the first peak were free C225, and the fractions corresponding to the second peak (fractions 14-21) were the desired conjugate (C225-PEG-PG-Adr), which was pooled, concentrated and stored at 4° C. As confirmed by GPC (system 2) analysis of purified C225-PEG-PG-Adr, the immunoconjugate was free of unconjugated C225 (FIG. 8). Specifically, FIG. 8 shows results of gel permeation chromatography of C225 (A), PEG-PG-Adr (B), and purified C225-PEG-PG-Adr conjugate (C) using a Superdex 200 column (1.0×30 cm). The compounds were monitored by measuring absorbance at 254 nm. Although C225-PEG-PG-Adr and PEG-PG-Adr was not completely resolved by GPC, the lack of tailing and the absence of a peak at 23.81 minutes in the chromatogram of C225-PEG-PG-Adr suggests that the product was free of unconjugated PEG-PG-Adr.

The elution curve of VS-PEG-PG-Adr had two peaks with retention times of 15.81 and 23.81 minutes, respectively. The first peak at 15.81 minutes in the GPC chromatogram of VS-PEG-PG-Adr appeared at the dead volume of the column, which may be attributed to the formation of polymer aggregates. The second peak may be attributed to the soluble form of the PEG-PG-Adr.

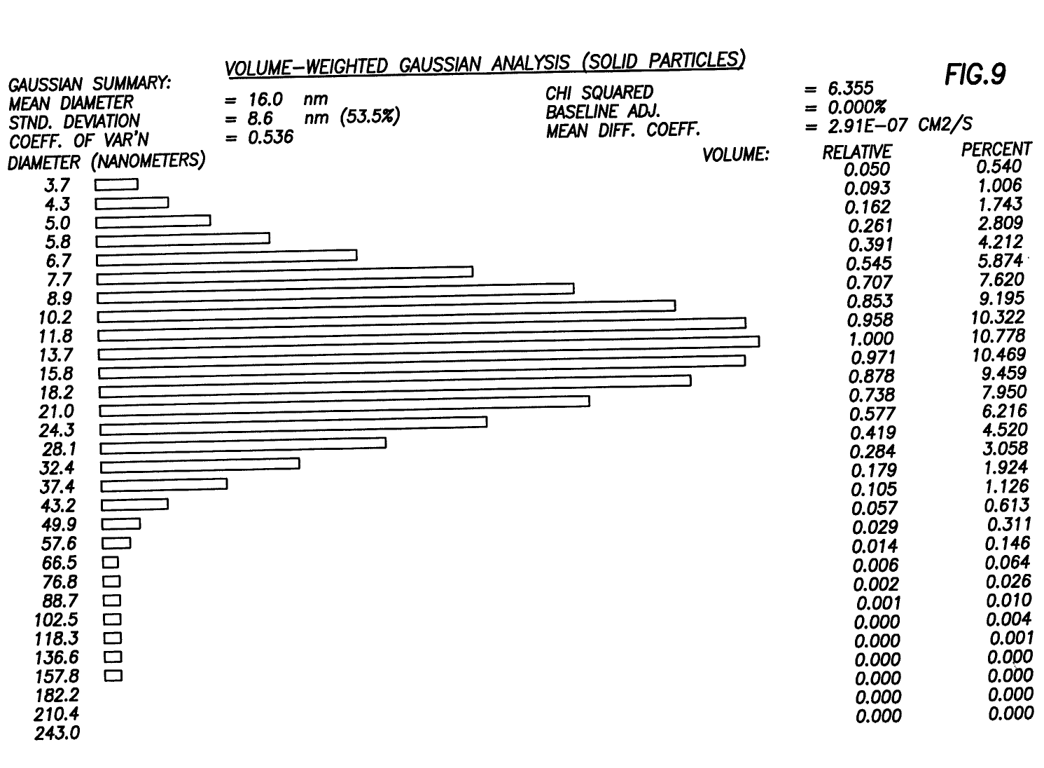

The nature of the polymeric aggregates may be attributed to the formation of nanoparticles with a hydrophobic core stabilized by outer hydrophilic PEG chains. Several lines of evidence support this conclusion. 1). PEG-poly(L-aspartic acid) (PEG-PAA) block copolymer with Adr coupled to the PAA has been shown to form micelles with average diameter of 40-60 nm (M. Yokoyama, et al, Preparation of micelle-forming polymer-drug conjugates. Bioconjugate Chem. 3:295-101, 1992). 2). The formation of particles with volume-average diameter of 207 nm was detected by light scattering. The size of the particles was decreased from 207 nm to 16 nm upon conjugation with C225 because of the increase in the hydrophilic segment of the amphiphilic block copolymer (Table 1, FIG. 9).

TABLE 1

| Compounds | dᵥₒₗᵤₘₑ (nm) |
|---|---|
| PEG-PG(31K)-Adr | 207 |
| PEG-PG(7.7K)-Adr | 121 |
| C225-PEG-PG(31K)-Adr | 16 |

On the other hand, decreasing the contribution of the hydrophobic block PG-Adr in PEG-PG-Adr by reducing the molecular weight of PG from 31K to 7.7K also resulted in reduction in particle size to 121 nm (Table 1). 3). PEG-PG-Adr did not form particles when it was dissolved in DMF.

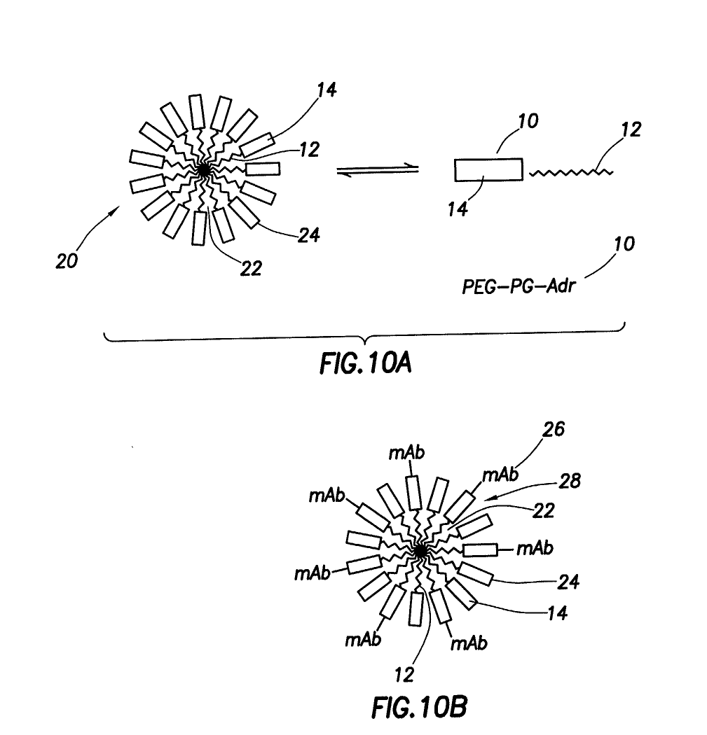

As shown in FIG. 10A, a block copolymer 10 (e.g., PEG-PG-Adr) composed of hydrophobic components 12 (e.g., PG-Adr) and hydrophilic components 14 (e.g., PEG) can form a nanoparticle structure 20 as a result of its amphiphilic character. The evidence indicates that PEG-PG-Adr adapted this structural feature, forming a plurality of nanoparticles 20. As shown in FIG. 10A, such nanoparticles 20 would consist of a hydrophobic PG-Adr core 22 surrounded by a hydrophilic outer PEG shell 24. As shown in FIG. 10B, one or more ligands 26 (e.g., mAb) may be attached to one or more of hydrophilic components 14, respectively, (e.g., PEG) to form targeted nanoparticle 28.

Attaching C225 to VS-PEG-PG-Adr affected the balance between hydrophilic and hydrophobic segments, resulting in decrease in particle size. Thus, the present invention describes a method to prepare targetable polymeric nanoparticles. Polymeric nanoparticles were obtained from a VS-PEG-PG-Adr copolymer. The VS functional groups residing on the surface of the nanoparticles provided a handle to further introduce homing moieties to the surface of the nanoparticles, whereas Adr attached to the core facilitated hydrophobic interactions to stabilize the nanoparticle structure.

### Example 3

### Biological Assays

Human vulvar squamous carcinoma A431 cells, human ovarian carcinoma SKOV-3 cells, or human breast cancer MDA-MB-468 cells were grown in DMEM-F12 medium containing 10% fetal bovine serum at 37° C.

a. Immunoprecipitation and Western Blotting Analysis

Cell pellets were treated with cold lysis buffer containing 1×protease inhibitor cocktails (Sigma, St Louis, Mo.) on soft ice for 30 minutes, followed by centrifugation to remove cell debris. Each test drug was added into 200 μl of supernatant in 0.5-ml microcentrifuge tubes. Two microliters of protein A beads (Sigma) were then added into each tube. The microcentrifuge tubes were incubated at 4° C. for 1 hour, centrifuged, and the beads washed 3 times with 0.5 ml lysis buffer. The beads were heated at 95° C. in 20 μl of 1×SDS-PAGE laemmli sample buffer (Bio-Rad, Hercules, Calif.) for 5 minutes, centrifuged, and analyzed by 7% SDS polyacrylamide gel electrophoresis (PAGE). Western blot was carried out by electronically transferring the samples into a nitrocellulose membrane and incubation of the membrane for 1 hour with an anti-EGF receptor antibody (Sigma) or anti-Her2/neu receptor antibody (Oncogen, Boston, Mass.). The receptor signals in the membrane were developed by the ECL chemoluminescence detection kit (Amersham Pharmacia Biotech Inc., Piscataway, N.J.).

Immunoprecipitation and western blot analysis were used to investigate the ability of Her-PEG-PG-Adr to bind to SKOV3 cells, which express a high level of Her2/neu receptors, as well as the ability of C225-PEG-PG-Adr to bind to A431 cells, which express a high level of EGF receptors. Both conjugates bound to their corresponding receptors in a dose-dependent manner with affinity similar to that of their parent antibodies. With respect to the A431 cells, a control of PEG-PG-Adr was also used; this control polymer without antibody did not bind to the receptors.

b. Intracellular Localization by Confocal Laser Microscopy

Confocal fluorescent microscope was used to investigate the binding of Her-PEG-PG-BODIPY to SKOV3 cells and C225-PEG-PG-Adr to A431 cells and their subsequent internalization. BODIPY (excitation/emission: 503/511 nm) and Adr (excitation/emission: 480/540-nm) were used to facilitate confocal fluorescent microscopic studies. Cells were grown on Lab-Tek II Chamber Slide (Nalge Nunc International, Naperville, Ill.) to 50% of confluence and incubated with immunoconjugates at 37° C. for various times. PEG-PG-Adr and PEG-PG-BODIPY were used as no-antibody polymer controls. MDA-MB-468 cells that do not express Her2/neu receptors were used as negative control when studying the binding of Her-PEG-PG-BODIPY to Her2/neu receptors. Cells were washed three times with PBS, fixed in 95% ethanol, and then treated with 1 μM TO-PRO-3 Iodide (Molecular Probes, Eugene, Oreg.) for 15 minutes for nuclei staining. Fluorescent images of cells were analyzed using LMS-510 confocal microscopy (Zeiss, Thornwood, N.Y.).

At one hour incubation confocal fluorescent microscopy images demonstrated that Her-PEG-PG-BODIPY, but not PEG-PG-BODIPY, selectively bound to SKOV3 cells overexpressing Her2/neu receptors. Furthermore, Her-PEG-PG-BODIPY did not bind to MDA-MB-468 cells that do not express Her2/neu receptors. It was not clear, however, whether the conjugates were internalized.

Similarly, confocal fluorescent microscopy images (at 15 minutes) demonstrated that C225-PEG-PG-Adr, but not PEG-PG-Adr, selectively bound to A431 cells. Unlike Herceptin conjugate, however, internalization of the C225-polymer conjugate was clearly visualized. C225-PEG-PG-Adr co-localized with nuclei, whereas PEG-PG-Adr without antibody was not localized to the nuclei. The process was very rapid, internalization was observed as early as 5 minutes after drug exposure. These results demonstrate that conjugation of a receptor-homing ligand to the end of a polymer chain through a PEG linker enhances the targeted delivery of therapeutic agents.

c. Cytotoxicity

One hundred microliter of growth medium suspending 1000-2000 cells per well was plated out in 96-well plates and incubated for 2 days to allow the cells to attach. Various dilutions of the drug or conjugates were added to each well and the plates were incubated for 72 hours at 37° C. Alternatively, a 6-hour pretreatment protocol was used. Cells were exposed to various concentrations of the drug or conjugate for 6 hours at 37 ° C., and then washed twice with the fresh culture medium. The cells were incubated for additional 72 hours. At the end of the incubation period, twenty microliters of MTT solution from Promega Cell Proliferation Assay kit (Madison, Wis.) were added to the wells. The microplates were then incubated for 1 hour at 37° C. Absorbance was measured at 490 nm using a microplate reader (Molecular Devices Corp, Sunnyvale, Calif.). The data reported represent the means of quadruplicate measurement and the standard errors of the mean were less than 15%. The IC₅₀, concentration exhibiting 50% growth inhibition were calculated from the growth-inhibition curve.

The cytotoxicities of Herceptin-conjugated PEG-PG-TXL and PEG-PG-TXL after 72 hours of continuous exposure were tested in two pairs of cells lines.

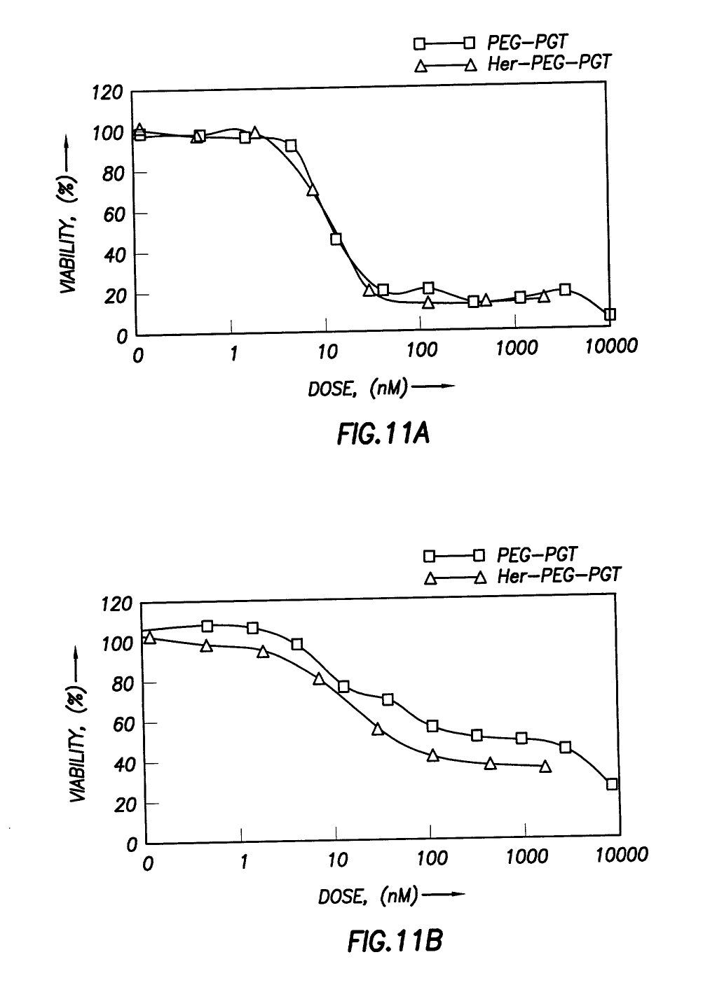

The first pair of cell lines included SKOV3ip1, a human ovarian cancer cell variant that overexpress Her2/neu, and MDA-MB-468, which does not express Her2/neu receptors. Results are shown in FIGS. 11A and 11B. The graph in FIG. 11A represents the MDA-MB-468 cells, and the graph in FIG. 11B represents the SKOV3ip1 cells. The y axis in each graph represents the % viability and the x axis represents the dose in nM. The data for PEG-PGT is represented by squares. The data for Her-PEG-PGT is represented by triangles.

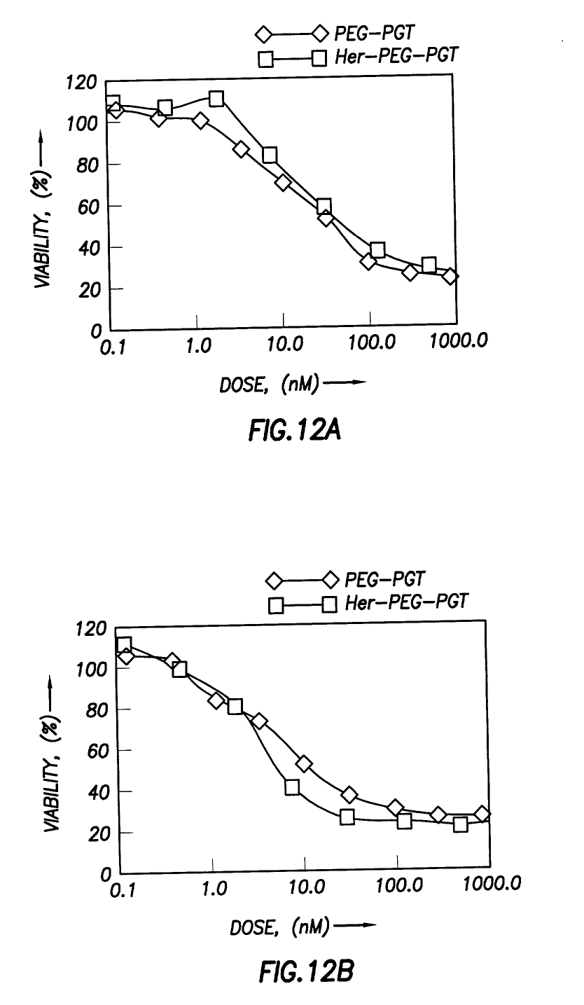

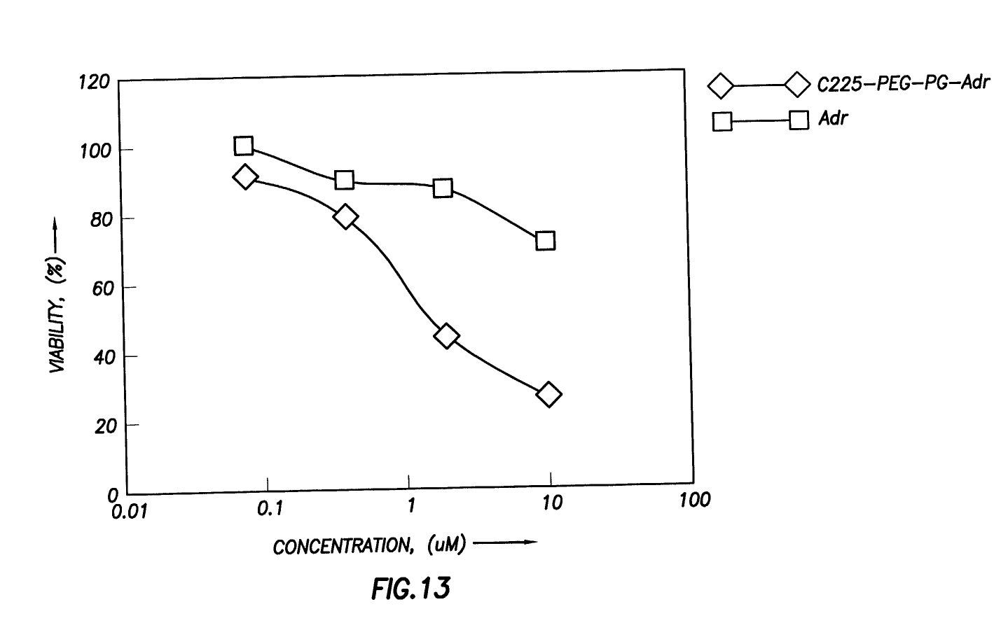

The second pair included MDA-MB-435 transfected with neo only (MDA435/neo), which does not express the receptor, and the stable Her2/neu transfectant MDA-MB435/eB2 (MDA 435/eB2). Results are shown in FIGS. 12A and 12B. The graph in FIG. 12A represents the MDA 435/neo cells; the graph in FIG. 12B represents MDA 435/eB2 cells. The y axis in each graph represents the % viability and the x axis represents the dose in nM. The data for PEG-PGT is represented by diamonds. The data for Her-PEG-PGT is represented by squares.

IC₅₀ values thus obtained were used to calculate targeting index, defined as the ratio of IC₅₀ values obtained with no-mAb drug conjugate PEG-PG-TXL in target cells and in non-target cells, times the ratio of IC₅₀ values obtained with mAb-conjugated PEG-PG-TXL in non-target cells and in target cells. The targeting index for the first pair and second pair of cell lines were 3.95 and 1.75, respectively. Since TXL is releasable from the immunoconjugates, one would expect that after 72 hours of incubation, a fraction of free TXL released from the conjugates could also contribute to the cytotoxic effect, resulting in reduced targeting index or selective cytotoxicity. It is anticipated that with a shorter incubation time, greater difference in potency between immunoconjugate and no-mAb polymer-drug conjugate would be observed.

To assess the specific cytotoxicity of the conjugate under conditions somewhat comparable to the in vivo situation, the cytotoxic activity was measured using a 6-hour pretreatment system. Confocal microscopic studies revealed that the binding of conjugate to cells reached maximum within 6 hours at 37° C. Under these conditions, the immunoconjugate C225-PEG-PG-Adr (IC₅₀ 1.69 μM) was 10-fold more potent than free Adr (IC₅₀ 17.7 μM). Results are shown in FIG. 13. In FIG. 13, data for C225-PEG-PG-Adr is represented by diamonds, and data for Adr is represented by squares. The y axis represents the % viability and the x axis represents the concentration in μg/ml. These data suggest that the binding and subsequent internalization of immunoconjugates significantly enhanced the cytotoxic activity of Adr.

d. Statistical Methods

Differences in cell growth inhibition were compared between different drugs using Student's t-test at the 0.05 significant level. Fluorescent intensity across the cells between different treatments were compared by repeat general linear model (p<0.05).

### Example 4

### Alternative Synthesis Method

As shown in this example, the homing ligand may also be introduced to the end of a PEG-PG block copolymer that contains a sulfhydryl group. The ligand is pretreated with γ-maleimidobutyric acid N-hydroxysuccinimide ester (GMBS) to introduce thio-reactive maleimide groups to the ligand.

a. Synthesis of SPDP-PEG-PNP

Into 245 mg NH₂-PEG-OH (MW 3,400, 0.072 mmol) in 3 ml of CH₂Cl₂ was added 10 μl triethylamine and 45 mg of N-succinimidyl 3-[2-pyridyldithio]propionate (SPDP) (0.144 mmol). The extent of the reaction was followed by ninhydrin spray test. To stop the reaction, the polymer was precipitated from CH₂Cl₂ with ethyl ether to give 233 mg of SPDP-PEG-OH (95%). The product was subsequently redissolved in 2 ml of CH₂Cl₂, and 17.5 mg (0.087 mmol) of p-nitrophenyl chloroformate (PNP) was added. The mixture was stirred at room temperature for 2 hours and the solvent was evaporated under vacuum. The residual material was precipitated and washed with ether to remove unreacted p-nitrophenyl chloroformate. Obtained 210 mg (86% from NH₂-PEG-OH).

b. Synthesis of SPDP-PEG-PG

The reaction followed procedures similar to those used for the synthesis of VS-PEG-PG. Briefly, PDP-PEG-pNP (100 mg, 0.029 mmol) was added into a solution of PG (200 mg, ˜0.0064 mmol) in 1 ml PBS (pH 8) in small portions over a period of 2 hours. The occurrence of the coupling reaction was evidenced by the release of yellowish p-nitrophenol into the reaction medium. The reaction was complete in 5 hours as revealed by ninhydrin test. The product was precipitated with 1 N HCl, dialyzed, and dried to afford 170 mg (86%).

c. Synthesis of SPDP-PEG-PG-Dox

Into a solution of 100 mg of SPDP-PEG-PG in 5 ml of DMF was added 25 mg of doxorubicin hydrochloride (Dox·HCl, 0.043 mmol) in 1 ml of DMF containing 20 μl of triethylamine. After being stirred for 15 min, 13 mg of coupling agent IIDQ (0.043 mmol) was added into the reaction mixture. The reaction was allowed to proceed at room temperature overnight. The solvent was evaporated under vacuum and the remaining solid was washed with ether, redissolved in 1 N NaHCO₃, dialyzed sequentially against PBS buffer (pH 7.2) and water, and lyophilized. Thin layer chromatography on silica using n-butanol:acetic acid:water (volume ratio 4:1:1) as mobile phase showed the absence of free Dox in the purified product. The conjugate contained 20% Dox (w/w) as determined by UV at 480 nm. Obtained conjugate 119 mg, the yield of Dox was 95%.

d. Synthesis of C225-PEG-PG-Dox

Four milligrams of dithiothreitol (DTT) was added into a solution of 10 mg SPDP-PEG-PG-Dox in 600 μl of PBS (pH 7.4) to obtain SH-PEG-PG Dox. The reaction was allowed to proceed at room temperature for 30 minutes followed by passing through a PD-10 column to remove unreacted DTT. In a separate reaction vessel, 50 mg of C225 was treated with γ-maleimidobutyric acid N-hydroxysuccinimide ester (GMBS) at a C225-to-GMBS molar ratio of 1:20. The reaction was stirred for 45 min and purified with a PD-10 column to remove small molecular weight contaminants. The solutions containing both SH-PEG-PG-Dox and maleimide-treated C225 were then mixed and stirred at room temperature for 2 hours. Unconjugated C225 was removed using a Resource Q anionic exchange column from a FPLC system as described above and concentrated with a Biomax-10000 Millipore ultracentrifuge with molecular-weight-cut-off of 10,000. The solution was desalted with a PD-10 column. One milliliter of C225-PEG-PG-Dox with a doxorubicin concentration of 0.8 mg/ml was recovered (40%). Blue precipitate (positive reaction) was observed when the Bio-Rad protein assay reagent was added into the conjugate solution, suggesting successful conjugation of C225 to PEG-PG polymer.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention. Not all embodiments of the invention will include all the specified advantages. The specification and examples should be considered exemplary only with the true scope and spirit of the invention indicated by the following claims.

We claim:

1. A conjugate molecule comprising:
a ligand;
a polymer spacer;
a polymer carrier; and
a therapeutic agent, wherein the ligand is bonded to the polymer spacer, the polymer spacer is bonded to the polymer carrier, and the polymer carrier is bonded to the therapeutic agent.

2. The molecule of claim 1, wherein the ligand is covalently bonded to the polymer spacer, the polymer spacer is covalently bonded to the polymer carrier, and the polymer carrier is covalently bonded to the therapeutic agent.

3. The molecule of claim 1, wherein the polymer carrier is bonded to the therapeutic agent with a linker.

4. The molecule of claim 1, wherein the ligand is an antibody, an antibody fragment, a peptide or a protein.

5. The molecule of claim 1, wherein the ligand is selected from the group consisting of C225, Herceptin, Rituxan, a phage library antibody, anti-CD, DC101, an antibody to integrin alpha v-beta 3, LM609, an antibody to VEGF, an antibody to VEGF receptor, F(ab′)₂, Fab′, ScFv fragment, c7E3Fab, a growth factor, VEGF-A, VEGF-B, VEGF-C, VEGF-D, PDGF, Angiopoietin-1, Angiopoietin-2, HGF, EGF, bFGF, cyclic CTTHWGFTLC, cyclic CNGRC, cyclic RGD-4C, annexin V, an interferon, a tumor necrosis factor, endostatin, angiostatin and thrombospondin.

6. The molecule of claim 1, wherein the ligand is an antibody.

7. The molecule of claim 1, wherein the ligand is a monoclonal antibody.

8. The molecule of claim 1, wherein the ligand is C225.

9. The molecule of claim 1, wherein the ligand is Herceptin.

10. The molecule of claim 1, wherein the ligand is c7E3Fab.

11. The molecule of claim 1, wherein the ligand is annexin V.

12. The molecule of claim 1, wherein the polymer spacer is selected from the group consisting of polyethylene glycol, a polyamino acid, polytyrosine,
polyphenylalanine, dextran, a polysaccharide, polypropylene oxide, a copolymer of polyethylene glycol with polypropylene oxide, polyglycolic acid, polyvinyl pyrolidone, polylactic acid and polyvinyl alcohol.

13. The molecule of claim 1, wherein the polymer spacer is polyethylene glycol.

14. The molecule of claim 13, wherein the polyethylene glycol has a number average molecular weight of about 1,000 daltons to about 100,000 daltons.

15. The molecule of claim 1, wherein the polymer carrier is selected from the group consisting of poly(1-glutamic acid), poly(d-glutamic acid), poly(d1-glutamic acid), poly(1-aspartic acid), poly(d-aspartic acid), poly(d1-aspartic acid), polylysine, a polysaccharide, polyhydroxypropylmethacryamide, dextran, poly(hydroxypropylglutamine), poly(hydroethylglutamine), hyaluronic acid, carboxymethyl dextran, polyacrylic acid, chitosan, and copolymers thereof.

16. The molecule of claim 1, wherein the polymer carrier is poly(1-glutamic acid).

17. The molecule of claim 16, wherein the poly(1-glutamic acid) has a number average molecular weight of about 1,000 daltons to about 100,000 daltons.

18. The molecule of claim 1, wherein the therapeutic agent is a chemotherapeutic agent.

19. The molecule of claim 18, wherein the chemotherapeutic agent is Adriamycin.

20. The molecule of claim 18, wherein the chemotherapeutic agent is paclitaxel.

21. The molecule of claim 1, wherein the therapeutic agent is selected from the group consisting of Adriamycin, daunorubicin, paclitaxel (Taxol), docetaxel (taxotere), epothilone, camptothecin, cisplatin, carboplatin, etoposide, tenoposide, geldanamycin, methotrexate and maytansinoid DM1, 5-FU, and gadolinium-DTPA.

22. A composition comprising a nanoparticle, said nanoparticle comprising a plurality of the conjugate molecules of claim 1.

23. A composition comprising the conjugate molecule of claim 1 and a pharmaceutically acceptable carrier.

24. The composition of claim 23, wherein the polymer carrier is bonded to the therapeutic agent with a linker.

25. The composition of claim 23, wherein the ligand is an antibody, an antibody fragment, a protein, or a peptide.

26. The composition of claim 23, wherein the ligand is an antibody.

27. The composition of claim 23, wherein the polymer spacer is PEG.

28. The composition of claim 23, wherein the polymer carrier is poly(1-glutamic acid).

29. The composition of claim 23, wherein the therapeutic agent is a chemotherapeutic agent.

30. The composition of claim 29, wherein the chemotherapeutic agent is Adriamycin or paclitaxel.

31. A method for selectively delivering a therapeutic agent to a target tissue in a patient comprising administering a conjugate molecule to the patient having said target tissue, wherein the conjugate molecule comprises: a ligand with affinity for the target tissue; a polymer spacer; a polymer carrier; and a therapeutic agent, wherein the ligand is bonded to the polymer spacer, the polymer spacer is bonded to the polymer carrier, and the polymer carrier is bonded to the therapeutic agent.

32. The method of claim 31, wherein the polymer carrier is bonded to the therapeutic agent with a linker.

33. The method of claim 31, wherein the ligand is an antibody, an antibody fragment, a protein, or a peptide.

34. The method of claim 31, wherein the ligand is an antibody.

35. The method of claim 31, wherein the polymer spacer is polyethylene glycol.

36. The method of claim 31, wherein the polymer carrier is poly(1-glutamic acid).

37. The method of claim 31 wherein the therapeutic agent is a chemotherapeutic agent.

38. The method of claim 31, wherein the administering step comprises intravascular, intraperitoneal or intramuscular injection.

39. The method of claim 31, wherein the patient is a mammal.

40. The method of claim 31, wherein the patient is a human.

41. The method of claim 31, wherein the target tissue is selected from the group consisting of a tumor, an inflammatory tissue, an infectious tissue, a reparative tissue and a regenerative tissue.

42. The method of claim 31, wherein the target tissue is a tumor.

43. The method of claim 42, wherein the tumor is a solid tumor.

44. The method of claim 42, wherein the tumor is breast cancer, ovarian cancer, colon cancer, lung cancer, head and neck cancer, brain cancer, liver cancer, pancreatic cancer, bone cancer, prostate cancer, lymphoma or leukemia.

45. A method of treating a patient having a diseased tissue, the method comprising administering a therapeutically effective amount of a conjugate molecule to the patient, wherein the conjugate molecule comprises: a ligand with affinity for the diseased tissue; a polymer spacer; a polymer carrier; and a therapeutic agent, wherein the ligand is bonded to the polymer spacer, the polymer spacer is bonded to the polymer carrier, and the polymer carrier is bonded to the therapeutic agent.

46. The method of claim 45, wherein the polymer carrier is bonded to the therapeutic agent with a linker.

47. The method of claim 45, wherein the ligand is an antibody.

48. The method of claim 45, wherein the polymer spacer is polyethylene glycol.

49. The method of claim 45, wherein the polymer carrier is poly(1-glutamic acid).

50. The method of claim 45, wherein the therapeutic agent is a chemotherapeutic agent.

51. The method of claim 45, wherein the administering step comprises intravascular, intraperitoneal or intramuscular injection.

52. The method of claim 45, wherein the patient is a mammal.

53. The method of claim 45, wherein the patient is a human.

54. The method of claim 45, wherein the diseased tissue is selected from the group consisting of a tumor, an inflammatory tissue, an infectious tissue, a reparative tissue and a regenerative tissue.

55. The method of claim 45, wherein the diseased tissue is a tumor

56. The method of claim 55, wherein the tumor is a solid tumor.

57. The method of claim 55, wherein the tumor is breast cancer, ovarian cancer, colon cancer, lung cancer, head and neck cancer, brain cancer, liver cancer, pancreatic cancer, bone cancer, prostate cancer, lymphoma or leukemia.

58. A method for synthesizing a conjugate molecule comprising the steps of:
providing a polymer spacer-polymer carrier construct having a sulfhydryl-reactive vinyl sulfone group at an end of the polymer spacer;
conjugating the therapeutic agent to the polymer carrier to form a vinyl sulfone-polymer spacer-polymer carrier-therapeutic agent construct;
pretreating the ligand to introduce sulfhydryl groups on the ligand; and
combining the pretreated ligand with the vinyl sulfone-polymer spacer-polymer carrier-therapeutic agent construct, wherein the vinyl sulfone group reacts with the sulfhydryl group to form said conjugate molecule comprising said ligand, said polymer spacer, said polymer carrier, and said therapeutic agent, and wherein the ligand is bonded to the polymer spacer, the polymer spacer is bonded to the polymer carrier, and the polymer carrier is bonded to the therapeutic agent.

59. A method for synthesizing a conjugate molecule comprising:
introducing a protected sulfhydryl group (SH) to an end of a polymer spacer;
conjugating the polymer spacer to a polymer carrier to form a protected SH-polymer spacer-polymer carrier construct;
conjugating a therapeutic agent to the polymer carrier to form a protected SH-polymer spacer-polymer carrier-therapeutic agent construct;
pretreating a ligand to introduce a sulfhydryl reactive functional group on said ligand;
deprotecting the protected SH group to obtain a free SH group; and
combining the pretreated ligand with the SH-polymer spacer-polymer carrier-therapeutic agent construct, wherein the SH group reacts with the sulfhydryl reactive functional group to form a conjugate molecule comprising the ligand, the polymer spacer, the polymer carrier, and the therapeutic agent, wherein the ligand is bonded to the polymer spacer, the polymer spacer is bonded to the polymer carrier, and the polymer carrier is bonded to the therapeutic agent.

60. The method of claim 59 wherein the ligand is pretreated with vinyl sulfone or maleimide to introduce the sulfhydryl reactive functional group.

61. A method for synthesizing a conjugate molecule comprising:
providing a polymer-spacer-polymer carrier-therapeutic agent construct;
introducing a protected amine to an end of the polymer spacer to form a protected amine-polymer spacer-polymer carrier-therapeutic agent construct;
deprotecting the protected amine-polymer spacer-polymer carrier-therapeutic agent construct to obtain a free amine-polymer spacer-polymer carrier-therapeutic agent construct; and
combining the free amine-polymer spacer-polymer carrier-therapeutic agent construct with a ligand having a carboxylic acid group, wherein the carboxylic acid in the ligand conjugates with the free amine to form an amide bond, thereby forming a conjugate molecule comprising the ligand, the polymer spacer, the polymer carrier and the therapeutic agent, wherein the ligand is bonded to the polymer spacer, the polymer spacer is bonded to the polymer carrier, and the polymer carrier is bonded to the therapeutic agent.
